(19) United States
(12) Patent Application Publication
(10) Pub. No.: US 2023/0089316 A1
(43) Pub. Date: Mar. 23, 2023

(54) BIOMATERIAL PRESERVING COMPOSITION

(71) Applicants: JBM Incorporation, Tokyo (JP); GN Corporation Co Ltd, Yamanashi (JP)

(72) Inventors: SHOJIRO KATOH, Tokyo (JP); SAMUEL JK ABRAHAM, Yamanashi (JP)

(21) Appl. No.: 17/801,102

(22) PCT Filed: Feb. 19, 2021
(86) PCT No.: PCT/JP2021/006281

§ 371 Date: Aug. 19, 2022

(30) Foreign Application Priority Data
Feb. 21, 2020 (JP) 2020-028227

(51) Int. Cl. A01N 1/02

(52) U.S. Cl. CPC A01N 1/0231

(57) ABSTRACT

A biomaterial preserving composition including a thermos-reversible polymer is provided. The composition is capable of solving problems related to conventional biomaterial preserving liquids not being sufficient for the survival or functional retention of cells in a biomaterial, and having many restrictions in preservation conditions such as the preservation temperature and the preservation treatment, resulting in a bottleneck for widespread use of cell therapy.

### CROSS-REFERENCE

This application is a national stage application, filed under 35 U.S.C. 371, of international application no. PCT/JP2021/006281, filed Feb. 19, 2021, which claims the benefit of Japanese Application No. 2020-028227, filed Feb. 21, 2020, each application of which is hereby incorporated herein by reference in its entirety.

### SEQUENCE LISTING

The instant application contains a Sequence Listing which has been submitted in ASCII format via EFS-Web and is hereby incorporated by reference in its entirety. Said ASCII copy, created on Oct. 23, 2020, is named PCT3120GN_SEQUENCE LISTING.txt and is 2000 bytes in size. The Sequence Listing is hereby incorporated herein by reference in it's entirety.

### TECHNICAL FIELD

The present invention relates to a biomaterial preserving composition comprising a thermoreversible gelation polymer.

### BACKGROUND

For recent years, cell therapies have been in practice for restoration of damaged tissue, etc. In order to perform a cell therapy, it is necessary that a certain number of viable cells are maintained in a biomaterial such as cells, a tissue or a cell sheet, and that they exert a desired function at a location where they are transplanted. Therefore, it is necessary to preserve the biomaterial during the time after being collected and processed until being transplanted. Methods that have been known so far for such preservation include a method of immersing the biomaterial in a preserving solution at a low temperature, a method for continuously perfusing a preserving solution, a method of preserving in a high-pressure gas, a method of preserving in an aerosol, etc. However, none of these methods for preserving biomaterial is sufficient in maintaining the viability or function of the cells in the biomaterial. Moreover, these methods have a number of restrictions on preserving conditions such as the preserving temperature and preserving processes, which has been a bottleneck in the prevalence of cell therapy.

### REFERENCES

[Patent Reference 1] JP A 2018-016654
[Patent Reference 2] WO 2010/049996
[Non-Patent Reference 1] Jamart et al., “Efficiency and limitation of Euro-Collins solution in kidney preservation”, J Surg Res. 1983 March; 34(3):195-204
[Non-Patent Reference 2] H. Yoshioka et al., “A Synthetic Hydrogel with Thermoreversible Gelation. I. Preparation and Rheological Properties”, Journal of Macromolecular Science, A31(1), 113-120 (1994)
[Non-Patent Reference 3] K. R. Holme. et al., “Chitosan derivatives bearing C10-alkyl glycoside branches: a temperature-induced gelling polysaccharide”, Macromolecules, 24, 3828-3833(1991)

### SUMMARY

### Problems to be Solved by the Invention

An object of the present invention is to provide a biomaterial preserving composition.

### Means to Solve the Problems

The present inventors have made an intensive research in order to solve the problems described above, and found that the viability and/or function of the cells in the biomaterial could be maintained by preserving the biomaterial using a thermoreversible gelation polymer, and thus reached the completion of the invention.

Namely, the present invention relates to the following biomaterial preserving compositions:

(1) A biomaterial preserving composition comprising a thermoreversible gelation polymer;
(2) The preserving composition according to (1), for preservation at a constant or varying temperature;
(3) The preserving composition according to (2), wherein the constant or varying temperature is a temperature at which a cell does not substantially proliferate;
(4) The preserving composition according to (3), wherein the temperature at which a cell does not substantially proliferate is 4 to 30° C.;
(5) The preserving composition according to any one of (1) to (4), wherein the biomaterial is selected from the group consisting of a cartilage tissue, oral mucosa tissue, corneal tissue, corneal limbus tissue, dental pulp tissue, vascular tissue, gastrointestinal mucosa tissue, greater omentum tissue, skin tissue and hepatic tissue;
(6) The preserving composition according to any one of (1) to (4), wherein the biomaterial is a somatic cell, precursor cell or stem cell contained in a tissue selected from the group consisting of a cartilage tissue, oral mucosa tissue, corneal tissue, corneal limbus tissue, dental pulp tissue, vascular tissue, gastrointestinal mucosa tissue, greater omentum tissue, skin tissue and hepatic tissue;
(7) The preserving composition according to any one of (1) to (6), wherein the thermoreversible gelation polymer comprising a plurality of blocks having cloud point selected from the group consisting of polypropylene oxide, a copolymer of propylene oxide and (an)other alkylene oxide(s), a poly N-substituted acrylamide derivative, a poly N-substituted methacrylamide derivative, a copolymer of an N-substituted acrylamide derivative and an N-substituted methacrylamide derivative, polyvinylmethylether and partially acetylated polyvinyl alcohol, and a hydrophilic block bound thereto; and,
(8) The preserving composition according to any one of (1) to (7), wherein the preserving composition can also be used for transportation.

### Effects of the Invention

The preserving composition of the present invention can maintain the viability and/or function of cells contained in a biomaterial better as compared with a conventional preserving solution. Furthermore, since the preservation does not have to be carried out at a constant low temperature, it is possible to preserve the biomaterial under a wide temperature condition. Moreover, because the preservation using the preserving solution of the present invention is less time-consuming and costs less, it can widely be utilized for a cell therapy, organ transplant, etc.

### BRIEF DESCRIPTION OF THE DRAWINGS

FIGS. 1A and 1B show Samples A and B for transporting a cartilage tissue, produced with either a thermoreversible gelation polymer (Sample A) or PBS (Sample B), respectively

FIG. 2 shows the expression level of COL2a1 which was expressed upon being cultured after being transported using a thermoreversible gelation polymer (Sample A) or ECS (Sample B).

FIGS. 3A-3J show the proliferative ability of a buccal mucosa after after being transported using a thermoreversible gelation polymer (Sample A) or ECS (Sample B) and cultured thereafter in plate-culture (DMEM and Cnt-PR) or using a thermoreversible gelation polymer (TGP).

FIGS. 4A-4D show corneal endothelial cells when each cornea was transported using either a thermoreversible gelation polymer (Sample A) or MK solution (Sample B).

FIGS. 5A-5D show corneal endothelial cells when each cornea was transported using either a thermoreversible gelation polymer (Sample A) or OptisolGS solution (Sample B).

FIGS. 6A and 6B show the proliferative ability of corneal limbuses after being transported using a thermoreversible gelation polymer (Sample A) or MK solution (Sample B) and cultured thereafter.

FIGS. 7A and 7B show the proliferative ability of an intestinal tissue after being transported using a thermoreversible gelation polymer (Sample A) or DMEM (Sample B) and cultured thereafter.

FIGS. 8A and 8B show the proliferative ability of a vascular tissue after being transported using a thermoreversible gelation polymer (Sample A) or M199 (Sample B) and cultured thereafter.

FIGS. 9A and 9B show the proliferative ability of a dental pulp tissue after being transported using a thermoreversible gelation polymer (Sample A) or DMEM (Sample B) and cultured thereafter.

FIG. 10 shows a HE staining image of a dental pulp tissue after being transported using a thermoreversible gelation polymer and cultured thereafter.

FIGS. 11A-11D show the proliferative ability of a skin tissue after being transported using a thermoreversible gelation polymer (Sample A) or HBSS (Sample B) and cultured thereafter.

FIGS. 12A and 12B show the proliferative ability of a hepatic tissue after being transported using a thermoreversible gelation polymer (Sample A) or DMEM (Sample B) and cultured thereafter.

### MODE FOR CARRYING OUT THE INVENTION

Unless otherwise defined herein, all technical terms and scientific terms used herein have the same meanings as usually understood by a person with ordinary skill in the art. All patents, applications, published applications and other publications referred herein are incorporated herein in their entirety by reference.

The biomaterial preserving composition of the present invention is characterized by that it comprises a thermoreversible gelation polymer.

### Biomaterials

In the present invention, a “biomaterial” means a cell, or a cell population, a cell culture, a structure, a tissue or an organ comprising the cell, and the cell is preferably a cell derived from a living organism, more preferably a primary cell obtained from an individual living organism. The cell may be a cell that has proliferated from a primary cell by culturing the primary cell for one or several passage(s). The cell may be a cell that had once proliferated by being cultured for several passages and that terminated the proliferation under certain biological or physical conditions. The cell encompasses, without being limited, for example, a somatic cell, as well as a sperm, an ovum, fertilized ovum and embryo. For example, the “cell” may be a primary cell or a established cell line. The cell includes, though not being limited thereto, those of liver (e.g., hepatic cell, sinusoidal endothelial cell), pancreas (e.g., pancreatic islet β cell), lung, brain (e.g., neural, glial or ependymal cell) or central or peripheral nervous system such as spinal cord, kidney, eye (e.g., retinal cell, corneal endothelial cell), spleen, skin, thymus, testis, lung, diaphragm, heart (heart cell), muscle or psoas muscle, or intestine (e.g., endocrine cell), adipose tissue (white, brown or beige adipocyte), muscle (e.g., fibroblast), synovicyte, chondrcyte, osteoclast, epithelial cell, endothelial cell, salivary gland cell, acoustic nerve cell, or hematopoietic cell (e.g., blood cell or lymphocyte), or a precursor cell and stem cell thereof (a tissue stem cell such as epithelial stem cell, satellite cell, intestinal stem cell, endothelial stem cell, olfactory mucosa stem cell, hair follicle stem cell, mammary gland stem cell, neural stem cell, hematopoietic stem cell, cardiac stem cell, and mesenchymal stem cell, embryonic stem cell, oocytes, blastomere, inner cell mass cell, embryonic germ cell, embryoid cell, morula-derived cell, teratomatous (teratocarcinoma) cell, and a multipotential, partly differentiated embryonic stem cell derived from the late embryogenesis stage, a pluripotent cells such as an iPS (induced pluripotent stem) cell.

The cell encompasses a tissue collected from a living organism or a cell population separated from an organ, etc. Cells that can be used in an embodiment of a cell population encompass, though not being limited thereto, an epithelial tissue-derived cell population (e.g., oral mucosa epithelial cells, epithelial stem cells, etc.), an adipose tissue-derived cell population (adipocytes, mesenchymal stem cells, etc.), a cartilage tissue-derived cell population (synovicytes, chondrcytes, chondrocyte-precursor cells, mesenchymal stem cells), as well as a cultured cell population, etc.

Furthermore, a “biomaterial” may include a cell culture or a cell structure. The cell culture or cell structure encompasses, without being limited, for example, a cell-polymer mixture, a cell sheet, and a cell cluster, whereas a cell structure encompasses, without being limited, a cell-polymer structure, a structure comprising a cell sheet, tissue, etc., bound therein.

The “biomaterial” is not limited as long as it comprises a cell, and may be a biological fluid including blood, bone marrow aspirate, lymph fluid, etc., and a tissue such as a thymus tissue, thyroid tissue, skeletal muscle tissue, tracheal tissue, vascular tissue, pulmonary tissue, hepatic tissue, gallbladder tissue, renal tissue, ureteral tissue, appendiceal tissue, bladder tissue, urethral tissue, testicular tissue, uterine tissue, ovarian tissue, gastrointestinal tissue (e.g., gastric tissue, small intestinal tissue or colorectal tissue), cardiac tissue, esophageal tissue, diaphragmatic tissue, splenic tissue, pancreatic tissue, brain tissue (e.g., cerebral tissue, cerebellar tissue), spinal cord tissue, cartilage tissue, extremity peripheral tissue, retinal tissue, skin tissue, oral mucosa tissue, corneal tissue, corneal limbus tissue, dental pulp tissue, vascular tissue, gastrointestinal tract tissue, greater omental tissue, skin tissue, hepatic tissue and amniotic membrane.

Moreover, the “biomaterial” may be an organ including salivary gland, palate, palatine uvula, tongue, tooth, pharynx, larynx, esophagus, liver, gallbladder, common bile duct, stomach, pancreas, pancreatic duct, small intestine (duodenum, jejunum, ileum), large intestine (transverse colon, ascending colon, cecum, descending colon, cecum, sigmoid colon, rectum), appendix, anus, heart, blood vessel, lymphatic vessel, lymph node, spleen, skin, thymus, nasal cavity, trachea, bronchus, lung, thorax, kidney, urinary duct, bladder, urethra, testis, uterus, ovary, fallopian tube, deferent canal, penis, vagina, eyeball, ear, brain, spinal cord, nerve fiber bundle, bone, cartilage, skeletal muscle, visceral muscle, tendon and ligament.

The biomaterial may be derived from any living organism. Such living organism includes, without limitation, such as, for example, human, non-human primates, dog, cat, pig, horse, goat, sheep, rodents (e.g., mouse, rat, hamster, guinea pig, etc.) and rabbit.

In the present invention, “preservation” means that the viability and/or function of cells contained in the biomaterial subjected to be preserved (hereinbelow the “biomaterial subjected to be preserved” will be referred to as the “biomaterial of interest”) is/are maintained. Maintenance of cell viability can be confirmed by a method that is usually employed when measuring the degree of the cell viability in the relevant technical field, such as, without limitation, measurement of the number or respiration activity of viable cells contained in the biomaterial of interest. It is preferred that the number or respiration activity of the viable cells contained in the biomaterial of interest is maintained, without limitation, by 70%, 60%, 50%, 40%, 30%, 20%, 10%, 5%, 1%, 0.1% or 0.05% after the preservation using the preserving composition of the present invention as compared to the level before the preservation. Maintenance of the functions can be confirmed by a method that is usually employed for measuring a function of a biomaterial in the relevant technical field, such as, without limitation, measurement of the proliferative ability of the cells contained in the biomaterial of interest, the maintenance of biomaterial morphology, an ability of secreting a prescribed component, or the expression level of a prescribed protein or gene. It is preferred that the function of the biomaterial of interest is maintained at the equal quality after the preservation using the preserving composition of the present invention as compared to the function before the preservation.

For instance, when the biomaterial of interest is a freshly collected cartilage tissue, its function may confirmed as the maintenance of the expression of a cartilage marker protein such as, without limitation, for example, SOX9, COL2A1, COL9A1, COL9A2, COL9A3, COL11A1, COL11A2, ACAN, HAPLN1, COMP or MATN3, or the gene encoding the same. Accordingly, in the present invention, a “preserving composition” means a composition to be employed in preservation, that is, a composition that has the aforementioned preserving effect when a biomaterial is immersed in it. The “preserving composition” encompasses both an embodiment which includes the biomaterial of interest and an embodiment which does not include the biomaterial of interest. As long as the viability and/or function of cells contained in the biomaterial of interest is/are maintained, the preservation not limited to be static, and includes any embodiment which might provide a vibration such as transportation. Accordingly, in one embodiment, the preserving composition of the present invention is a composition for transportation.

### Thermoreversible Gelation Polymer

The thermoreversible gelation polymer (also referred herein as “TGP”) contained in the preserving composition of the present invention refers to a polymer that has a characteristic of being capable of producing a cross-linked structure or meshwork structure in a thermoreversible manner, and, based on this structure, producing in a thermoreversible manner a hydrogel which retains a separating solution such as water. The preserving composition of the present invention has the characteristic of this polymer. The hydrogel refers to a gel comprising a cross-linked or meshwork structure consisting of polymers and water supported or retained in this structure.

Sol-Gel Transition Temperature

In the present invention, definitions and measurement of a “sol state”, a “gel state” and a “sol-gel transition temperature” are based on the definitions and measurement described in Non-Patent Reference 2 (H. Yoshioka et al., “A Synthetic Hydrogel with Thermoreversible Gelation. I. Preparation and Rheological Properties”, Journal of Macromolecular Science, A31(1), 113-120 (1994)). That is, the dynamic elastic modulus of a sample at the observation frequency of 1 Hz is measured while gradually changing the temperature from the lower side to the higher side (1° C./1 min), and the temperature at which the storage elastic modulus (G′, elastic item) of the sample exceeds the loss elastic modulus (G″, viscosity item) is defined as the sol-gel transition temperature. In general, a sol is defined as the state of G″>G′, whereas gel is defined as the state of G″<G′. For measurement of this sol-gel transition temperature, the following measuring conditions can suitably be used.

Conditions for Measuring Dynamic/Loss Elastic Modulus
Measuring apparatus (trade name): Stress-controlled rheometer AR500 (TA Instruments)
Sample solution (or separating solution) concentration (as the concentration of a “hydrogel-forming polymer having a sol-gel transition temperature”: 0(weight) %
Amount of the sample solution: Approximately 0.8 g
Shape and dimensions of the measuring cell: Acrylic parallel disk (diameter: 4.0 cm), gap: 600 μm
Measuring frequency: 1 Hz
Applied stress: Inside the linear region.

In the present invention, the sol-gel transition temperature of the thermoreversible gelation polymer is preferably higher than 0° C. and not higher than 37° C., and further preferably higher than 5° C. and not higher than 35° C. (in particular, equal to or higher than 10° C. and equal to or less than 33° C.). Since the preserving composition of the present invention can preserve the biomaterial of interest whether it is in a sol state or in a gel state, its sol-gel transition temperature may be, without limitation, between 10 and 35° C., or between 15 and 30° C. From a viewpoint that by being gelled it can prevent the preservation media being agitated by the vibration at the time of preservation or transportation and can suppress the stress by agitation, the sol-gel transition temperature is preferably between 17 and 25° C., more preferably between 19 and 23° C., particularly preferably between 19 and 21° C., where the gelation can occur at a normal room temperature. A TGP having such suitable sol-gel transition temperature can easily be selected from specific compounds mentioned hereinafter according to the screening method (sol-gel transition temperature measuring method) described above.

The TGP of the present invention is not limited as long as it exhibits thermoreversible sol-gel transition as described above (that is, it has a sol-gel transition temperature). As specific examples of a polymer whose aqueous solution has a sol-gel transition temperature and which indicates in a reversible manner a sol state at a temperature that is lower than the transition temperature, for example, a polyalkylene oxide block copolymer represented by a block copolymer of polypropylene oxide and polyethylene oxide, etc.; an etherified cellulose such as methylcellulose and hydroxypropylcellulose; and a chitosan derivative (K. R. Holme. et al. “Chitosan derivatives bearing C10-alkyl glycoside branches: a temperature-induced gelling polysaccharide”, Macromolecules, 24, 3828-3833 (1991) (Non-Patent Reference 3)), etc.

Suitable Thermoreversible Gelation Polymers

A hydrogel-forming polymer which utilizes a hydrophobic binding for crosslink formation and which can suitably be used as the TGP of the present invention is preferably composed of a plurality of blocks having cloud point and a hydrophilic block binding together. The presence of this hydrophilic block is preferable because the hydrogel will become water-soluble at a temperature that is lower than the sol-gel transition temperature. On the other hand, in the presence of a plurality of blocks having cloud point, the hydrogel will change into a gel state at a temperature that is higher than the sol-gel transition temperature.

In other words, because a block having cloud point will be dissolved in water at a temperature that is lower than the cloud point and will change into insoluble at a temperature that is higher than the cloud point, it plays a role as a crosslinking point constituted by a hydrophobic binding for gel formation at a temperature that is higher than the cloud point. Namely, the cloud point derived from the hydrophobic binding corresponds to the sol-gel transition temperature of the hydrogel described above. It is noted that the cloud point and the sol-gel transition temperature are not necessarily be consistent. This is because the cloud point of the above-described “block having cloud point” is generally influenced by the binding of the block and a hydrophilic block. Owing to such characteristics, the biomaterial of interest can be immersed in the preserving composition comprising a thermoreversible gelation polymer at a lower temperature. Accordingly, from a viewpoint of preventing the biomaterial of interest from being damaged, a thermoreversible gelation polymer comprising a plurality of blocks having cloud point and a hydrophilic block bound thereto is preferred.

The hydrogel used in the present invention utilizes its characteristic not only that the strength of its hydrophobic binding is increased as temperature rises but also that this change is reversible against temperature. The TGP preferably has a plurality of “blocks having cloud point” from viewpoint of that more than one crosslinking points will be formed within one molecule, resulting in a formation of a gel that has a superior stability, thereby increasing the preservability of the biomaterial of interest. On the other hand, the hydrophilic block in the TGP described above has a function, as mentioned before, that it changes the TGP to be water-soluble at a temperature that is lower than the sol-gel transition temperature, preventing the above-described hydrogel from being aggregated and deposited due to an excess increase in the strength of its hydrophobic binding at a temperature that is higher than the above-described transition temperature, while forming a state of a water-containing gel. Moreover, it is desirable that the TGP used in the present invention is degraded and taken up in vivo. That is, the TGP of the present invention is preferably degraded in vivo by hydrolytic or enzymatic reaction, taken up and excreted as a biologically non-toxic, low molecular-weight substance.

When the TGP of the present invention comprises a plurality of blocks having cloud point and a hydrophilic block bound thereto, it is preferred that at least one, preferably both of the blocks having cloud point and the hydrophilic block is/are degraded and taken up in vivo.

Plurality of Blocks Having Cloud Point

The block having cloud point is preferably a polymer block whose solubility-temperature coefficient against water indicates a negative value. More specifically, a polymer selected from a group consisting of polypropylene oxide, a copolymer of propylene oxide and (an)other alkylene oxide(s), a poly N-substituted acrylamide derivative, a poly N-substituted methacrylamide derivative, a copolymer of an N-substituted acrylamide derivative and an N-substituted methacrylamide derivative, polyvinylmethylether and partially acetylated polyvinyl alcohol can preferably used. A poly N-substituted acrylamide derivative, poly N-substituted methacrylamide derivative, and a copolymer with N-substituted methacrylamide derivative are preferred in view of that a gel with excellent stability will be formed and thereby the preservability of the biomaterial of interest will be increased.

In order that the block having cloud point is to be degraded and taken up in vivo, it is effective that the block having cloud point is a polypeptide composed of a hydrophobic amino acid and a hydrophilic amino acid. Alternatively, a polyester-type biodegradable polymer such as polylactic acid or polyglycolic acid can also be utilized as the block having cloud point that is degraded and taken up in vivo.

The cloud point of the above-described polymer (the block having cloud point) is preferably higher than 4° C. and not higher than 40° C. from a viewpoint of that this will make the sol-gel transition temperature of the polymer used in the present invention (a compound comprising a plurality of blocks having cloud point and a hydrophilic block bound thereto) to be higher than 0° C. and not higher than 37° C. Here, measurement of the cloud point can be carried out, for example, by refrigerating a 1% by mass aqueous solution of the above-described polymer (the block having cloud point) to form a clear homogeneous solution, then gradually raising the temperature (temperature-raising rate: approx. 1° C./min), and deciding the point at which the solution first becomes clouded as the cloud point.

Specific examples of the poly N-substituted acrylamide derivative, poly N-substituted methacrylamide derivative that can be used in the present invention are listed below:

poly-N-acryloyl piperidine; poly-N-n-propyl methacrylamide; poly-N-isopropyl acrylamide; poly-N,N-diethyl acrylamide; poly-N-isopropyl methacrylamide; poly-N-cyclopropyl acrylamide; poly-N-acryloyl pyrrolidine; poly-N,N-ethylmethyl acrylamide; poly-N-cyclopropyl methacrylamide; poly-N-ethylacrylamide.

The polymer described above may be a homopolymer, or may be a copolymer of a monomer and (an)other monomer(s) that constitute the polymer described above. As other monomers that constitute such copolymer, either of a hydrophilic monomer or hydrophobic monomer can be used. In general, the polymerization with a hydrophilic monomer will increase the cloud point of a product, whereas the polymerization with a hydrophobic monomer will decrease the cloud point of the product.

Accordingly, a polymer that has a desired cloud point (e.g., a cloud point that is higher than 4° C. and not higher than 40° C.) can also be obtained by selecting a monomer to be copolymerized.

Hydrophilic Monomer

The hydrophilic monomer described above includes N-vinylpyrrolidone, vinylpyridine, acrylamide, methacrylamide, N-methyl acrylamide, hydroxyethyl methacrylate, hydroxyethyl acrylate, hydroxymethyl methacrylate, hydroxymethyl acrylate, an acrylic acid and a methacrylic acid having an acidic group, and a salt thereof, vinyl sulfonic acid, styrene sulfonic acid, etc., as well as N,N-dimethyl aminoethyl methacrylate, N,N-diethyl aminoethyl methacrylate and N,N-dimethyl aminopropyl acrylamide having a basic group and salts thereof, although not being limited thereto.

Hydrophobic Monomer

On the other hand, the hydrophobic monomer described above includes an acrylate derivative and methacrylate derivative such as ethyl acrylate, methyl methacrylate and glycidyl methacrylate, an N-substituted alkyl methacrylamide derivative such as N-n-butyl methacrylamide, vinyl chloride, acrylonitrile, styrene, and vinyl acetate, although not being limited thereto.

Hydrophilic Block

On the other hand, the hydrophilic block to be bound to the block having cloud point described above include, in specific, methylcellulose, dextran, polyethylene oxide, polyvinyl alcohol, poly N-vinylpyrrolidone, polyvinylpyridine, polyacrylamide, polymethacrylamide, poly N-methyl acrylamide, polyhydroxy methyl acrylate, poly acrylic acid, poly methacrylic acid, polyvinylsulfonic acid, polystyrene sulfonic acid and salts thereof; poly N,N-dimethyl aminoethyl methacrylate, poly N,N-diethyl aminoethyl methacrylate, poly N,N-dimethyl aminopropyl acrylamide and salts thereof.

Moreover, it is desirable that the hydrophilic block is degraded, metabolized and excreted in vivo. Therefore, hydrophilic biomacromolecules including proteins such as albumin and gelatin, polysaccharides such as hyaluronic acid, heparin, chitin and chitosan are favorably used.

The method for binding the block having cloud point and the hydrophilic block described above is not particularly limited, though it can be carried out, for example, by introducing into one of the blocks described above a polymerizable functional group (e.g., acryloyl group) and copolymerizing thereto a monomer that will give the other block. Moreover, the binding product of the block having cloud point and the hydrophilic block described above can also be obtained by block copolymerization of a monomer that will give the block having cloud point and a monomer that will give the hydrophilic block. The binding of the block having cloud point and the hydrophilic block can also be carried out by introducing into both blocks a reaction-active functional group (e.g., hydroxyl group, amino group, carboxy group, isocyanate group, etc.) in advance and binding both blocks via a chemical reaction. In that case, usually a plurality of reaction-active functional groups are introduced into the hydrophilic block. Moreover, the binding of the polypropylene oxide having cloud point and the hydrophilic block can be carried out, for example, by repeated sequential polymerization of propylene oxide and a monomer that constitutes “(an)other hydrophilic block(s)” (e.g., ethylene oxide) through anionic or cationic polymerization, thereby yielding a block copolymer in which polypropylene oxide and a “hydrophilic block” (e.g., polyethylene oxide) are bound. Such block copolymer can also be obtained by introducing into an end of polypropylene oxide a polymerizable group (e.g., acryloyl group) and then copolymerizing thereto a monomer that constitutes the hydrophilic block. Furthermore, the polymer used in the present invention can also be obtained by introducing into the hydrophilic block a functional group that is capable of binding to a functional group (e.g., hydroxyl group) at an end of the polypropylene oxide, and reacting them. Moreover, the TGP used in the present invention can also be obtained by coupling a material in which polyethylene glycol is bound to both end of polypropylene glycol such as Pluronic® F-127 (Trade name, ADEKA CORPORATION).

The polymer of the present invention in this embodiment comprising a block having cloud point is completely dissolved in water and indicates a sol state at a temperature that is lower than the cloud point, because the above-described “block having cloud point” present within the polymer is water-soluble together with the hydrophilic block. However, when an aqueous solution of this polymer is heated to a temperature that is higher than the cloud point described above, the “blocks having cloud point” present within the molecule become hydrophobic and the association of blocks between separate molecules occur due to hydrophobic interaction. On the other hand, because the hydrophilic block is still water-soluble at this time (when being heated to a temperature that is higher than the cloud point), the polymer of the present invention, in water, generates a hydrogel which has a three-dimension meshwork structure in which the hydrophobic association part between the blocks having cloud point is a crosslinking point. When the temperature of this hydrogel is again cooled to a temperature that is lower than the cloud point of the “blocks having cloud point” present within the molecule, the blocks having cloud point becomes water-soluble, releasing the crosslinking point due to the hydrophobic association and eliminating the hydrogel structure, making the TGP of the present invention a complete aqueous solution again. In such a way, the sol-gel transition of the polymer of the present invention in a preferred embodiment is based on the change in the reversible hydrophilicity and hydrophobicity of the blocks having cloud point that are present within the molecule at the cloud point, and therefore, it has a complete reversibility corresponding to a temperature change. In one embodiment of the present invention, because the association occurs between separate molecules due to such hydrophobic interaction, the gel will not be dissolved by tissue fluid, etc., from the biomaterial of interest, and the environment of the biomaterial of interest can be maintained constant. A delicate balance of hydrophilicity-hydrophobicity of the above-described TGP in water can contribute to the stability of the biomaterial of interest when it is preserved.

Gel Solubility

The hydrogel-forming polymer of the present invention comprising at least a polymer having a sol-gel transition temperature in an aqueous solution as mentioned above indicates substantially water-insoluble at a temperature that is higher than the sol-gel transition temperature (d ° C.) and indicates water-soluble in a reversible manner at a temperature that is lower than the sol-gel transition temperature (e ° C.).

The above-described high temperature (d ° C.) is preferably a temperature that is higher than the sol-gel transition temperature by 1° C. or more, further preferably a temperature that is higher by 2° C. or more (in particular, by 5° C. or more). Moreover, being “substantially water-insoluble” as described above is preferably that the amount of the above-described polymer that can be dissolved in 100 milliliter of water at the above-described temperature (d ° C.) is 5.0 g or less (furthermore 0.5 g or less, 0.1 g or less, in particular).

On the other hand, the above-described low temperature (e ° C.) is preferably a temperature that is lower than the sol-gel transition temperature by 1° C. or more (by absolute value), further preferably a temperature that is lower by 2° C. or more (in particular, 5° C. or more). Moreover, being “water-soluble” as described above is preferably that the amount of the above-described polymer that can be dissolved in 100 milliliter of water at the above-described temperature (e ° C.) is 0.5 g or more (furthermore 1.0 g or more). Furthermore, “indicating water-soluble in a reversible manner” refers to that the aqueous solution of the above-described TGP indicates water-soluble as described above at a temperature that is lower than the sol-gel transition temperature, even once after being gelled (at a temperature that is higher than the sol-gel transition temperature).

The polymer described above preferably indicates a viscosity of 10 to 3,000 centipoise (furthermore, 50 to 1,000 centipoise) at 5° C. in 10% aqueous solution. Such viscosity is preferably measured, for example, under a condition as follows:
Viscometer: Stress-controlled rheometer (Model name: AR500, TA Instruments)
Rotor diameter: 60 mm
Rotor shape: Parallel disk

When the aqueous solution of the TGP of the present invention is gelled at a temperature that is higher than the above-described sol-gel transition temperature, the resulting gel will not substantially be dissolved even when being immersed in a large amount of water. This characteristic of the hydrogel formed by the above-described TGP can be confirmed, for example, as follows.

Namely, 0.15 g of the TGP is dissolved in 1.35 g of distilled water at a temperature that is lower than the above-described sol-gel transition temperature (e.g., on ice) to generate a 10 wt % aqueous solution, which is poured into a plastic Petri dish of 35 mm diameter and heated to 37° C. to form a gel of 1.5 mm thick in the Petri dish, and then the weight of the whole Petri dish including the gel (f gram) is measured. Next, the whole Petri dish including the gel is left still in 250 milliliter of water at 37° C. for 10 hours, and then the weight of the whole Petri dish including the gel (g gram) is measured and whether the gel has been dissolved or not from the surface of the gel is evaluated. In this case, in the hydrogel-forming polymer of the present invention, the rate of weight decrease of the gel, i.e., (f−g)/f, is preferably 5.0% or less, further preferably 1.0% or less (in particular, 0.1% or less).

When the aqueous solution of the TGP of the present invention is gelled at a temperature that is higher than the above-described sol-gel transition temperature, the resulting gel will not be dissolved for a prolonged period even when being immersed in a large amount (as much as 0.1 to 100 times the gel in volume ratio) of water. Such characteristic of the polymer used in the present invention is achieved, for example, by the presence of two or more (a plurality) of blocks having cloud point withing the polymer.

The present inventors has found that, on the other hand, when a similar gel is generated using the aforementioned Pluronic® F-127 comprising polyethylene oxide being bound to both end of polypropylene oxide, the gel will completely be dissolved in water after being left still in water for a few hours.

In order to suppress cytotoxicity during non-gelling time as low as possible, it is preferred to use a TGP that is capable of being gelled at a concentration in water, i.e., a concentration of {(polymer)/(polymer+water)}×100(%) of 20% or less (furthermore, 15% or less, in particular, 10% or less).

The molecular weight of the TGP used in the present invention is preferably equal to or more than 30,000 and equal to or less than 30,000,000, more preferably equal to or more than 100,000 and equal to or less than 10,000,000, further preferably equal to or more than 500,000 and equal to or less than 5,000,000.

TGP Concentration, Etc. In Preserving Composition

The TGP in the preserving composition of the present invention may be dissolved in any medium. The TGP in the preserving composition may be at any concentration as long as the viability of the cells contained in the biomaterial of interest is maintained, and may be, without limitation, as percent concentration by mass, 1 to 40%, 3 to 30%, 5 to 20%, 7 to 15%, 8 to 12%, or 9 to 11%. It is preferably at 7 to 15%, 8 to 12%, or 9 to 11% from a viewpoint of that the TGP in a sol state has a viscosity of such a degree that the biomaterial of interest can be suspended without being in contact with the bottom surface of the preservation container, and preferably comprises 9 to 11% TGP from a viewpoint of that the TGP can, upon being gelled, form a cross-linked or meshwork structure having a similar degree of pressure as the pressure under which the biomaterial of interest was present in vivo.

The TGP contained in the preserving composition of the present invention may be dissolved in any medium. The medium is not particularly limited as long as the viability of the cells can be maintained, though, typically, those which are based on a physiological saline, various physiological buffer solution (e.g., PBS, HBSS, etc.), various basal medium for cell culturing, preserving solution, or transporting solution can be used. The compositions of the physiological saline and various physiological buffer solution may be altered as appropriate according to the biomaterial of interest and other preserving conditions.

The basal medium includes, without limitation, for example, DMEM, MEM, F12, DME, RPM11640, MCDB (MCDB102, 104, 105 (M199), 107, 120, 131, 153, 199, etc.), L15, SkBM, RITC80-7, and CnT-PR, etc. Many of these basal media are commercially available and their compositions are known. The basal medium may be use in its standard composition (e.g., as being sold in market), or its composition may be altered as appropriate according to the cell species and cell conditions. A preserving solution includes, without limitation, for example, EPII solution, MK solution and OptisolGS solution; a tissue or organ transporting solution includes, without limitation, choline solution, Euro-Collins solution, UW solution, HTK solution, Celsior solution, Polysol and Dsol, etc. The physiological saline, basal medium, preserving solution and transporting solution used as a medium of the present invention are not limited to those of known compositions, and include those to which one or more ingredients are added, removed, increased or decreased.

The medium may include one or more additives apart from those described above, such as a serum, growth factor (e.g., EGF, insulin, etc.), steroid component or selenium component. In one embodiment of the present invention, the medium that dissolves the TGP does not comprise a serum. In one embodiment of the present invention, the medium that dissolves the TGP may comprise a serum. The serum may be a xenogeneic serum or allogeneic serum. A allogeneic serum is preferred, among which autologous serum is particularly preferred. The serum can be contained in the medium at a concentration of, without limitation, 1% or higher, 3% or higher, 5% or higher, 10% or higher, or 20% or higher. The concentration is preferably 10%.

The preserving composition of the present invention may further comprise any additional ingredient unless it compromises the preserving effect of the preserving composition. The additional ingredient may include, for example, an acceptable carrier, any ingredient that increases the viability of a cell culture (vitamins, amino acids, etc.), antibiotics and antiseptics. Any known ingredient can be used as such additional ingredient, and a person with ordinary skill in the art is familiar with these additional ingredients. An ingredient that can enhance the effect of the preserving composition of the present invention is preferred.

In the present invention, the state of the TGP may be a sol state or a gel state as long as the biomaterial of interest can be preserved. From the viewpoint of being capable of suppressing the agitation during transportation, preservation in a gel state is preferred. For preservation in a sol state, typically, the biomaterial of interest is immersed in the TGP sol at a low temperature, without raising the temperature of the sol thereafter. For preservation in a gel state, typically, the biomaterial of interest is immersed in the TGP sol, then the sol can be heated to a temperature that is higher than the sol-gel transition temperature to become to a gel state.

In one embodiment of the present invention, after immersing the cells, etc. to be preserved in the TGP sol and gelling the sol, a medium may further be added onto the TGP. This is preferable for a long-term preservation because it is thus possible to provide nutrients into the TGP through the medium. The medium to be added onto the gelled TGP may be the same as or different from the medium in which the TGP is dissolved. The medium to be added onto the TGP is preferably the same as the medium in which the TGP is dissolved except that the later does not comprise a serum.

Preserving Temperature

The temperature for preserving the biomaterial of interest may be, without limitation, for example, between 1° C. and 42° C., between 4° C. and 38° C., between 6 and 35° C., between 10 and 35° C., between 12 and 30° C., between 15 and 30° C., between 20 and 30° C., between 22 and 28° C., between 23 and 27° C., or between 24° C. and 26° C., as long as the viability of the cells in the biomaterial of interest can be maintained. It is preferably between 15 and 30° C., more preferably between 20 and 30° C. From the viewpoint that preventing the biomaterial of interest from being damaged due to temperature change, preservation at a constant temperature is preferred, though the composition can be used in order to preserve the biomaterial of interest at a varying temperature. Accordingly, in one embodiment of the present invention, the preserving temperature may be constant or varying. The temperature may vary without limitation as long as the viability of the cells in the biomaterial of interest can be maintained, though it may typically vary in a range between 1° C. and 42° C., between 4° C. and 38° C., between 6 and 35° C., between 10 and 35° C., between 12 and 30° C., between 15 and 30° C., between 20 and 30° C., between 22 and 28° C., between 23 and 27° C., or between 24° C. and 26° C., preferably varies in a range between 15 and 30° C., between 20 and 30° C., and more preferably in a range between 20 and 30° C. In one embodiment of the present invention, the varying temperature may be the outdoor temperature, which may diurnally vary in the aforementioned range of the temperature variation.

In one embodiment of the present invention, the preserving temperature may be a temperature at which the cells, etc., to be preserved will not substantially proliferate. The temperature at which the biomaterial of interest “will not substantially proliferate” may be a temperature at which the proliferation of a cell will be slowed or stopped in the relevant technical field, i.e., a temperature at which the proliferation rate or respiration rate of the biomaterial of interest is ⅓, ⅕, 1/10, 1/20, or 1/100, or smaller as compared to the proliferation rate or respiration rate at 37° C. The temperature may be, without limitation, for example, 30° C. or lower, and may be 27° C. or lower, 25° C. or lower, 23° C. or lower, 20° C. or lower, 17° C. or lower, 15° C. or lower, 12° C. or lower, 10° C. or lower, 7° C. or lower, 6° C. or lower, or 4° C. or lower. Specifically, for example, it may be between 4 and 30° C., between 6 and 30° C., between 8 and 30° C., between 10 and 30° C., between 12 and 30° C., between 14 and 30° C., between 16 and 30° C., between 18 and 30° C. or between 20 and 30° C., preferably between 16 and 30° C., and particularly specifically between 20 and 30° C.

In one embodiment of the present invention, the preserving temperature may be constant or may vary at a temperature at which the cells, etc. to be preserved will not substantially proliferate.

Preserving Time

The preserving time may not be limited as long as the biomaterial of interest can be preserved, and may be, as an upper limit, for example, 1 hour or longer, 3 hours or longer, 5 hours or longer, 12 hours or longer, 18 hours, 24 hours or longer, 2 days or longer, 4 days or longer, 8 days or longer, 12 days or longer, 16 days or longer, 20 days or longer, 30 days or longer, 40 days or longer, 50 days or longer, or 60 days or longer, and includes, as a lower limit, for example, up to 45 days, up to 35 days, up to 25 days, up to 14 days, up to 10 days, up to 6 days, up to 3 days, up to 20 hours, up to 16 hours, up to 14 hours, up to 10 hours, up to 8 hours, up to 6 hours, up to 5 hours, up to 4 hours, 3 hours, up to 2 hours, up to 1 hour, etc. The preserving time may be any combination of these upper and lower limits, and includes, without limitation, a range between 3 hours and 60 days, between 6 hours and 50 days, between 8 hours and 40 days, between 10 hours and 25 days, between 12 hours and 20 days, between 18 hours and 15 days, between 24 hours and 10 days, or between 36 hours and 8 days. The biomaterial of interest should not preferably be preserved for a long time because a damage will proceed by a long time preservation.

Since the preserving composition of the present invention has a superior preservability of a biomaterial, it can suitably be utilized, without limitation, for example, in a field of medicine or medical experiment, particularly as a composition for preserving or transporting a biomaterial used for cell therapy or organ transplant.

### EXAMPLES

### Production Example 1

Forty-two grams of N-isopropyl acrylamide and n-butyl methacrylate 4.0 g were dissolved in 592 g of ethanol. To this, an aqueous solution of 11.5 g of polyethylene glycol dimethacrylate (PDE6000, NOF CORPORATION) dissolved in 65.1 g of water was added and heated to 70° C. under nitrogen gas stream. While maintaining at 70° C. under nitrogen gas stream, 0.4 mL of N,N,N′,N′-tetramethylethylene diamine (TEMED) and 4 mL of 10% aqueous solution of ammonium persulfate (APS) were added, stirred for 30 minutes to allow for reacting. Furthermore, 0.4 mL of TEMED and 4 mL of 10% aqueous solution of APS were added four times in thirty-minute intervals to complete the polymerization reaction. The reaction solution was cooled to 5° C. or lower, then diluted by adding 5 L of cooled distilled water at 5° C., and concentrated to 2 L using a ultrafiltration membrane of molecular cutoff=100,000 at 5° C.

The concentrated solution was diluted by adding 4 L of cooled distilled water, and the ultrafiltration operation described above was performed again. The dilution and the ultrafiltration operation described above were repeated five more times to remove anything with a molecular weight of 100,000 or less. Those which were not filtrated by this ultrafiltration (those which remained within the ultrafiltration membrane) were collected and lyophilized to yield 40 g of a hydrogel-forming polymer of the present invention having a molecular weight of 100,000 or higher (“hydrogel-forming polymer”-6). One gram of the hydrogel-forming polymer of the present invention obtained as described above (“hydrogel-forming polymer”-6) was dissolved in 9 g of distilled water on ice to yield a 10 wt % aqueous solution. The storage elastic modulus of this aqueous solution was 43 Pa at 10° C., 680 Pa at 25° C., and 1310 Pa at 37° C., as being measured using a stress-controlled rheometer (AR500, TA Instruments) at an application frequency of 1 Hz. This temperature-dependent change in the storage elastic modulus was observed repeatedly in a reversible manner. The sol-gel transition temperature was approximately 20° C.

### Example 1: Transportation of Cartilage Tissue

A part of a cartilage tissue resected by joint replacement (approx. 10×5 mm, 3 mm thick, age: 35) was sampled and immersed in PBS containing antibiotics (gentamicin (50 μg/ml), amphotericin (0.25 μg/ml), penicillin (100 Units/ml)/streptomycin (100 μg/ml)) for 30 minutes. The cartilage tissue was cut with a scalpel into tissue slices of approximately 3 mm³. Then, 1 g of the TGP produced in Production Example was dissolved in 9 ml of DMEM at 4° C. to generate a 10% TGP solution. To the generated TGP solution, the tissue slices were added, which was uniformly dispersed by pipetting in immersion. The flask was left still at room temperature to allow for gelation, then 7 to 8 ml of 10% serum-containing DMEM medium (Thermo Fisher Scientific, DMEM, high glucose, Cat NO:11965-084) was added, and the tissue slices were cultured in 5% carbon dioxide incubator (ESPEC BNA-111). The medium was replaced every week for 42 days of culturing. After 42 days, PBS was added at 4° C. to the TGP gel in which the tissue slices had been embedded, and the TGP gel was dissolved by pipetting. The tissue slices were transferred to a 50 ml test tube. To the test tube, 20 ml of PBS was added at 4° C., which was then centrifuged and washed, and this was repeated twice. The resulting tissue slices were divided into two, each was weighed and then added into two 10 ml test tubes (Test Tubes A and B).

Sample A: One gram of the TGP produced in Production Example was dissolved in 9 ml of DMEM at 4° C. to generate a 10% TGP solution, of which 10 ml was added to Test Tube A containing a tissue slice, gelled at 30° C. for 1 hour, then transported at a temperature varying between 5 and 42° C. (outdoor temperature) for 3 hours. The weight of the tissue slice was 0.23 g (n=4).

Sample B: Ten milliliters of PBS (phosphate buffered solution) was added to Test Tube B containing a tissue slice, which was transported at 4° C. for 3 hours. The weight of the tissue slice was 0.16 g (n=4).

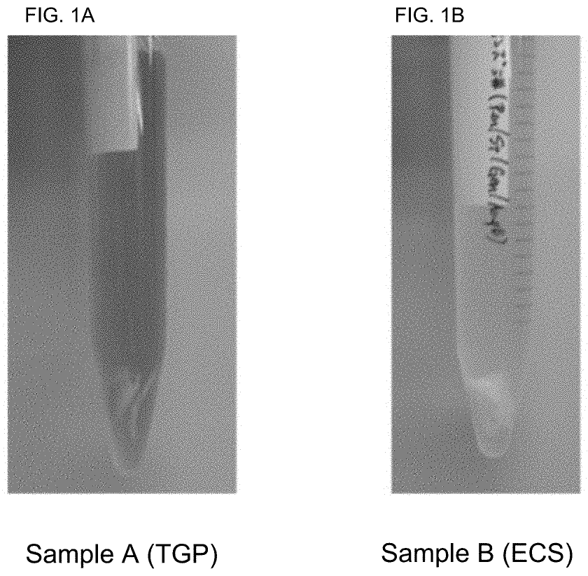

Photographs of Samples A and B containing cartilage tissue slices are shown in FIGS. 1A and 1B. The tissue slices of Samples A and B were washed with 4° C. PBS, then each was treated with Trypsin-EDTA solution (0.25%) at 37° C. for 30 minutes, then digested with Collagenase II solution (1 mg/ml) at 37° C. for 19 hours. After being filtrated through a 100 μm cell-strainer (PLS, Cat No: 43-50100-03), it was centrifuged (150 rpm, 5 minutes) and suspended in 2 ml PBS. From each test tube, 100 μl was separately collected, to which 400 μL of 0.4% trypan blue solution was added, and the cells were counted using a cytometer. The results are shown in Table 1.

TABLE 1

| | | Viable | Relative |
|---|---|---|---|
| | Tissue | Cell | Viable |
| No | Weight | Count | Cell Count |
| Sample A (TGP) | 0.23 g | 10.0 × 10⁴ | 4.4 × 10⁵/g |
| Sample B (PBS) | 0.16 g | 4.0 × 10⁴ | 2.5 × 10⁵/g |

It was found that a high viable cell count and a high relative viable cell count were maintained when being transported using the TGP as compared with the case when being transported using PBS. From this result, it was found that the TGP is suitable for transportation of a cartilage tissue.

### Example 1-2: Transportation of Cartilage Tissue

Cartilage tissue slices were prepared according to the same procedures as in Example 1-1 except using a cartilage tissue from another specimen, and added to Test Tubes A and B.

Sample A: One gram of the TGP produced in Production Example was dissolved in 9 ml of DMEM at 4° C. to generate a 10% TGP solution, of which 10 ml was added to Test Tube A containing a tissue slice, gelled at 30° C. for 1 hour. BioBox (SUGIYAMA-GEN CO., LTD., CatNo.SBE-10W) and Thermostrage 20 (SUGIYAMA-GEN CO., LTD., CatNo. TP-20-350) were used to keep the temperature at approximately 20° C. during 3 hours of transportation. The weight of the tissue slice contained in Sample A was 0.21 g.

Sample B: Ten milliliter of Euro-Collins solution (Corning Glucose Solution (Euro-Collins), Cat No: 99-408-CM) (hereinbelow, Euro-Collins solution is referred to as “ECS”) was added to Test Tube B containing a buccal tissue slice, which was transported at 4° C. for 3 hours. The weight of the tissue slice contained in Sample B was 0.19 g.

Each tissue slice was cut with scalpel into slices of 1 mm² or smaller. These tissue slices were treated with Trypsin-EDTA solution (0.25%) at 37° C. for 30 minutes, then digested with Collagenase II solution (1 mg/ml) at 37° C. for 12 to 16 hours. This was washed with DMEM solution, then filtered through a filter (100 μm), and then centrifuged (1800 rpm, 10 minutes). The precipitate was diluted with 10% serum-containing DMEM solution, then added into a T25 flask and cultured in 5% carbon dioxide incubator. The culture solution was replaced every three days for 2 weeks culturing. After 2 weeks, culture supernatant was discarded, and the cells were dispersed with Trypsin-EDTA (0.25%) solution.

One gram of the TGP produced in Production Example was dissolved in DMEM at 10° C. to generate a 10% TGP solution. The cells derived from the cartilage tissue slice were dispersed in this solution and dispensed into a 6-well plate. After leaving at room temperature for gelation, 7 to 8 ml of a medium (10% serum-containing DMEM solution containing antibiotics (gentamicin (50 μg/ml), amphotericin (0.25 μg/ml), penicillin (100 Units/ml)/streptomycin (100 μg/ml)) and L-Ascorbic acid (5 mg/ml)) was added, and the cells were cultured in 5% carbon dioxide incubator (ESPEC BNA-111). The culture solution was replace every week for 4 to 16 weeks culturing.

The cartilage cell cultures obtained by the culturing were collected at Day 42. Using RNeasy Mini Kit (Qiagen), mRNAs were isolated from the collected cultures. From the obtained total RNAs, 1 μg was used as template to synthesize cDNAs by reverse-transcription using Superscript III reverse transcriptase (Invitrogen). TB Green Premix Ex Taq II (Takara, Cat No. RR820S/A/B) was used for Real-time PCR analysis, Thermal Cycler Dice Real Time System II (Takara, Cat No. TP900) was used for measurement. The primers used were shown below:

SOX9: Fwd
(SEQ ID NO: 1)
5′-ggagatgaaatctgttctgggaatg-3′
SOX9: Rvs
(SEQ ID NO: 2)
5′-ttgaaggttaactgctggtgttctg-3′
COL2A1: Fwd
(SEQ ID NO: 3)
5′-ccagttgggagtaatgcaagga-3′
COL2A1: Rvs
(SEQ ID NO: 4)
5′-acaccaggttcaccaggttca-3′

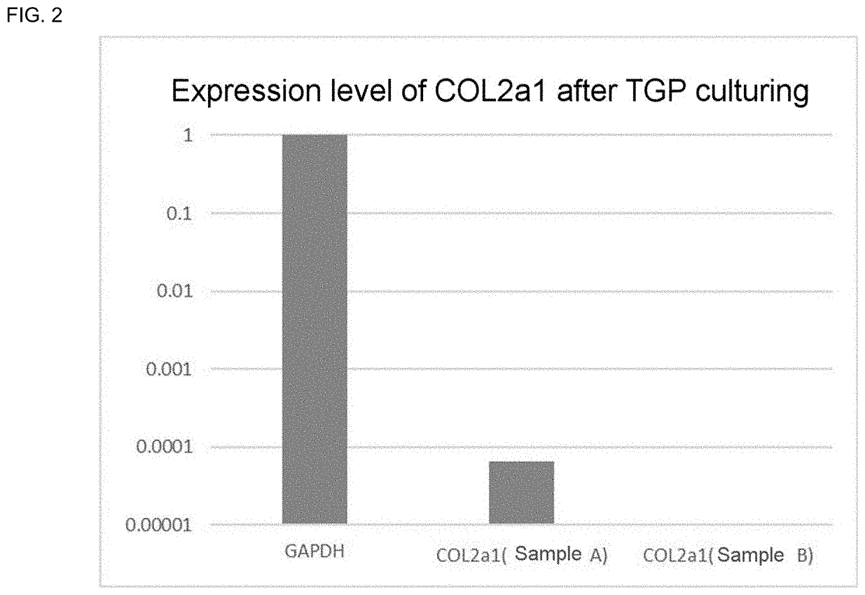

From the results of Real-time PCR, SOX9 was expressed at similar level in both Samples A and B. On the other hand, COL2A1 was not expressed in Sample B (ECS transportation) on Day 42, whereas it was expressed in Sample A (TGP transportation) on Day 42. The results for COL2A1 were shown in FIG. 2.

It was found that, when being transported using the TGP, SOX9 and COL2A1, which are known to be markers for a healthy cartilage tissue were expressed even 42 days after culturing. Together with the results from Example 1-1, it was found that the transportation using the TGP had a good influence not only on the viable cell count but also on the characteristics of the cultured tissue thereafter as compared with the case of being transported using PBS or ECS. From this result, it was found that the TGP is suitable for transportation of a cartilage tissue.

### Example 2-1: Transportation of Oral Tissue

Using the same procedures as in Example 1, oral mucosa tissue (3 mm³) was collected from oral cavities of human subjects (Age: 54 (#1080), Age: 21 (#1081), Age: 17 (#1082), Age: 36 (#1083)). Four samples were collected from each subject, and washed. Each tissue slice was equally cut into two pieces, each added into one of the two 10 ml test tube (Samples A and B).

Transportation of Tissue

Sample A: One gram of the TGP produced in Production Example was dissolved in 9 ml of DMEM at 4° C. to generate a 10% TGP solution, of which 10 ml was added to Test Tube A containing a tissue slice, gelled at 30° C. for 1 hour. BioBox (SUGIYAMA-GEN CO., LTD., CatNo.SBE-10W) and Thermostrage 20 (SUGIYAMA-GEN CO., LTD., CatNo. TP-20-350) was used to keep the temperature at approximately 20° C. during 4 hours of transportation.

Sample B: Ten milliliter of PBS was added into Test Tube B containing a buccal tissue slice, which was transported at 4° C. for 4 hours.

Measurement of Viable Cell Count

The tissue slices of Samples A and B were washed with 4° C. PBS, then each was weighed to be 0.05 mg, treated with Trypsin-EDTA solution (0.25%) at 37° C. for 30 minutes, then digested with Collagenase II solution (1 mg/ml) at 37° C. for 2 hours. After being filtrated through a 100 μm cell-strainer, it was centrifuged (150 rpm, 5 minutes), suspended in 2 ml PBS. From each sample, 100 μl was separately collected, to which 400 μL of 0.4% trypan blue solution was added, and the cells were counted using cytometer. The results are shown in Table 2.

TABLE 2

| | Cell Count After | |
|---|---|---|
| Sample | Transport (×10⁶) | |
| No. | Sample A | Sample B |
| #1080 | 0.87 | 0.45 |
| #1081 | 0.46 | 0.11 |
| #1082 | 0.32 | 0.18 |
| #1083 | 0.41 | 0.22 |

Results

Since all of the epithelial tissues transported using the TGP were confirmed to contain many viable cells, it was found that the preservation using the TGP is suitable for transportation of an epithelial tissue.

### Example 2-2: Transportation of Oral Tissue

A part of a healthy buccal tissue slice from the oral cavity of a human subject (Age: 34) (#1084) was collected and transported according to the same procedures as in Example 2 except using ECS (Euro-Collins solution, Corning Glucose Solution (Euro-Collins), Product Number 99-408-CM) instead of PBS.

Conforming Proliferative Ability

Each tissue slice from Samples A and B was washed with PBS at 4° C. One gram of the TGP produced in Production Example was dissolved in 9 ml of culture solution to generate a 10% TGP, and each tissue slice from Samples A and B was seeded therein. After gelation at 30° C. for 1 hour, the culture solution was added, and the tissues were cultured in 5% carbon dioxide incubator (Sample A-TGP, Sample B-TGP). The proliferative ability of the cells in culture was confirmed using an inverted microscope.

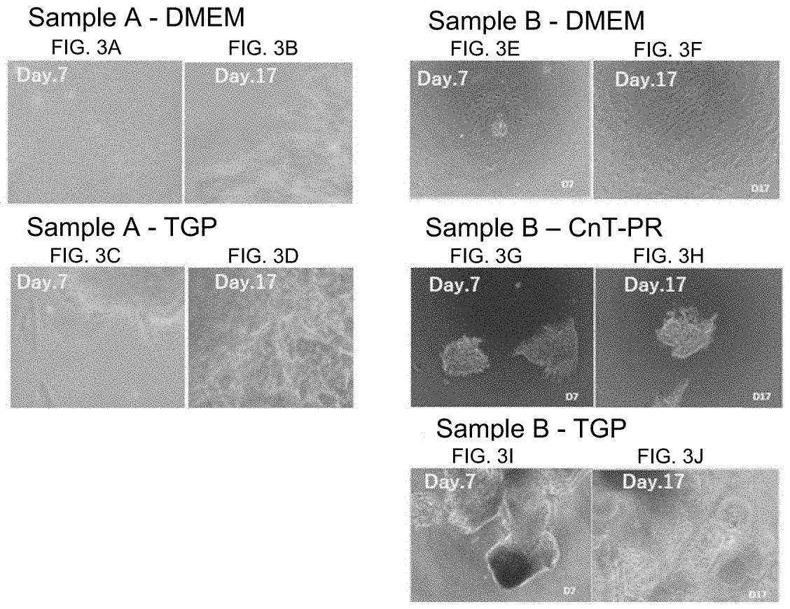

A part of the tissue slices from Samples A and B was added into a 25 cm² flask with DMEM, which was then cultured (Sample A-DMEM, Sample B-DMEM). Furthermore, a part of the tissue slices of Sample B was added into a 25 cm² flask containing 10 ml of Cnt-PR, which was then cultured (Sample B-Cnt-PR). The inverted microscope images of Sample A-DMEM, Sample A-TGP after culturing for 7 days or 17 days are shown in FIGS. 3A-3J (left). Sample B-DMEM, Sample B-CnT-PR, Sample B-TGP are shown in FIGS. 3A-3J (right). Note that the magnification is 10 in all images, except for 40 for Sample A-TGP and Day 17 of Sample B-TGP.

When the tissue slice from Sample A was cultured using DMEM or TGP, cell proliferation was confirmed at a relatively early stage (Sample A-DMEM, -TGP). As shown in FIGS. 3A-3J show, in Sample A-DMEM, it was indicated that a number of cells had proliferated from the tissue slice after 17 days of culturing. The fastest proliferation was confirmed in Sample A-TGP among all samples. In Sample A-TGP, a very large number of cells had proliferated by the time after 17 days of culturing.

When the tissue slice from Sample B was cultured using DMEM, Cnt-PR or TGP (Sample B-DMEM, -Cnt-PR, -TGP), the proliferation rate is slow in all cases, though it was relatively fast in the case being cultured using the TGP among these. As shown in FIGS. 3A-3J, in Sample B-DMEM, it was indicated that a small number of cells had proliferated from the tissue slice, though, fibroblasts started to grow from the tissue slice after 7 days of culturing, which covered the bottom of the culture plate after 17 days of culturing. In Sample B-Cnt-PR, a small number of cells had proliferated by the time after 17 days of culturing.

In Sample B-TGP, may cells had proliferate from the tissue slice by the time after 17 days of culturing. Because epithelial cells proliferated well in Sample A regardless of culture solution, it was found that the transportation using TGP would cause less damage to the tissue as compared with ECS.

On Day 21 of culturing, the tissue slice from Sample A-TGP, Sample A-DMEM, Sample B-TGP and Sample B-DMEM was each washed with PBS at 4° C. The tissue slices of Samples A and B were washed with 4° C. PBS, then each treated with Trypsin-EDTA solution (0.25%) at 37° C. for 30 minutes, then digested with Collagenase II solution (1 mg/ml) at 37° C. for 5 hours. After being filtrated through a 100 μm cell-strainer, it was centrifuged (150 rpm, 5 minutes), suspended in 2 ml PBS. From each sample, 100 μl was separately collected, to which 400 μL of 0.4% trypan blue solution was added, and the cells were counted using a cytometer. The results are shown in Table 3.

TABLE 3

| | Cell Count after | |
|---|---|---|
| | Culture (×10⁶) | |
| Culture Method | Sample A | Sample B |
| TGP Culture | 0.77 | 0.44 |
| | E:N = 28:72 | E:N = 43:57 |
| Plate Culture | 0.45 | 0.12 |
| | E:N = 73:27 | E:N = 62:38 |

E:N in the table indicates the ratio of epithelial cells to non-epithelial cells.

The highest cell count was shown by Sample A transported with the TGP followed by culturing with the TGP. The lowest cell count was shown by Sample B transported with ECS followed by plate culturing. Moreover, Sample B, which showed a low viable cell count after being transported in Example 2, confirmed a sufficient viable cell count when being cultured with the TGP. From this, it was found that the TGP increases the proliferation ability of the cells in the epithelial tissue, and particularly restores the proliferation ability of the epithelial tissue in which the viable cell count has been decreased.

Moreover, it was elucidated that culturing with the TGP increases the ratio of non-epithelial cells in all cases. An epithelial cell has a morphology that resembles a polygonal paving stone, whereas a non-epithelial cell has a round morphology which is a characteristic of an epithelial stem cell. The proliferated non-epithelial cells were therefore considered to be epithelial stem cells. Thus, it was also found that culturing of an epithelial tissue using TGP propagates epithelial stem cells in the epithelial tissue.

### Example 3-1: Transportation of Cornea (MK Solution)

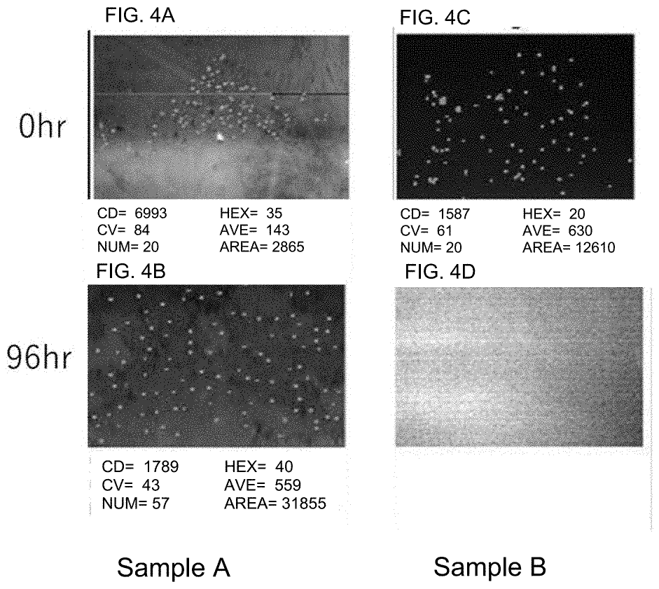

Eye bolls were collected from human body remains, immersed in sterilized 0.5% solution of I-PVP (iodine-polyvinylpyrrolidone) for 2 minutes. Two corneas were collected according to a conventional method, immersed in PBS containing antibiotics (gentamicin (50 μg/ml), amphotericin (0.25 μg/ml), penicillin (100 Units/ml)/streptomycin (100 μg/ml)) for 30 minutes. Then, one gram of the TGP produced in Production Example was dissolved in 9 ml of MK solution (5% Dextran 40-containing M199 (Thermo Fisher cat no. 11150-067) at 4° C. to generate a 10% TGP solution. One of the two corneas was transferred to a cornea-preserving container, and the TGP solution was added such that the whole cornea was soaked, gelled at 30° C. for 1 hour, and 10 ml of MK solution was added. This was transported at 5 to 42° C. for 96 hours (Sample A). The other of the corneas was transferred to a cornea-preserving container, to which 10 ml of MK solution was added (Sample B), maintained at 4° C. during 96 hours of transportation. Corneas of Samples A and B were observed before preservation and after 96 hours transportation using a specular microscope (Keratoanalyzer EKA-10, KONAN MEDICAL, INC.) and the software (KSS-EB10, KONAN MEDICAL, INC.). The results are shown in FIGS. 4A-4D. The top two photographs show before-preservation (0 hour), and the bottom two photographs show the corneal tissues after 96 hours transportation.

It was found that there were a large number of good quality endothelial cells, which is a characteristic of a cornea, in Sample A after preservation for 96 hours. On the other hand, in Sample B, no viable endothelial cell was confirmed after 96 hours. Thus, as compared with Sample B, the cornea was preserved well in Sample A. Accordingly, it was elucidated that the TGP is suitable for transporting a cornea.

### Example 3-2: Transportation of Cornea (OptisolGS Solution)

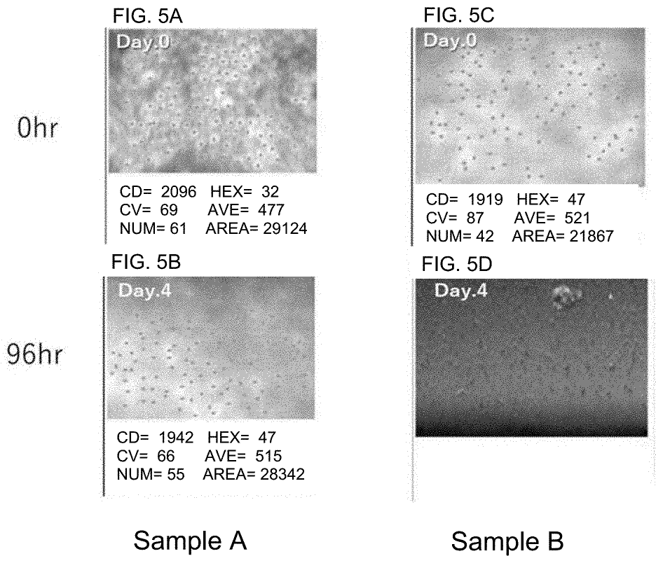

From another specimen, two corneas were collected according to the same procedures as in Example 3-1. Samples A and B were prepared and transported according to the same procedures as in Example 3-1 except using OptisolGS solution (OptiSol-GS Corneal Storage Media (Box of 12) (Bausch & Lomb 50006-OPT)) instead of MK solution used in Example 3-1. Corneas of Samples A and B were observed before preservation and after 96 hours transportation using a specular microscopy (Keratoanalyzer EKA-10, KONAN MEDICAL, INC.) and the software (KSS-EB10, KONAN MEDICAL, INC.). The results are shown in FIGS. 5A-5D. The top two photographs show before-preservation (0 hour), and the bottom two photographs show the corneal tissues after 96 hours transportation. It was elucidated that there were a large number of good quality endothelial cells, which is a characteristic of a cornea, in Sample A after preservation for 96 hours. On the other hand, in Sample B, no viable endothelial cell was confirmed on Day 4. Thus, as compared with Sample B, the cornea was preserved well in Sample A. Accordingly, it was elucidated that the TGP is suitable for transporting a cornea.

### Example 4-1: Transportation of Corneal Limbus

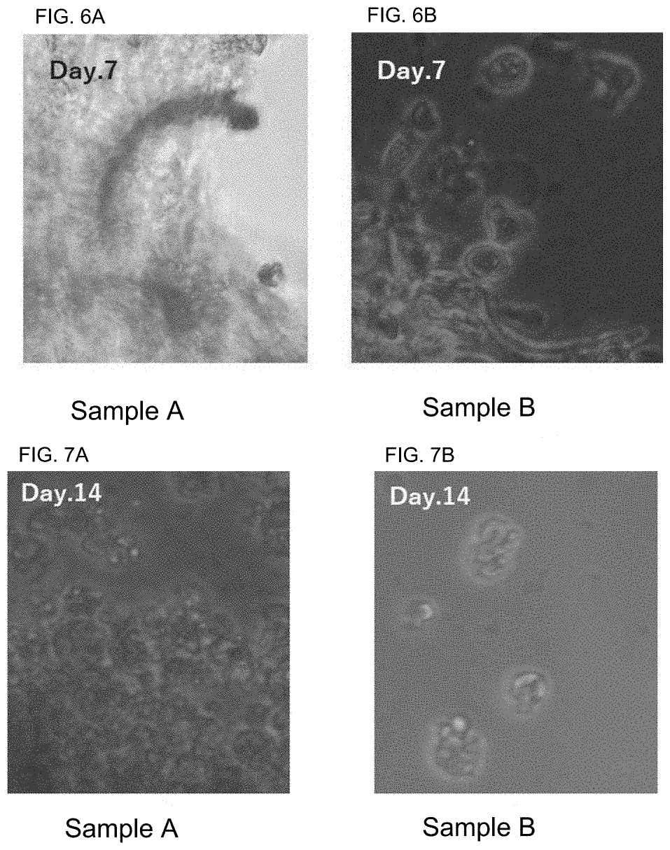

From another specimen, corneal limbus tissue slices were collected instead of corneas. Samples A and B were prepared according to the same procedures as in Example 3-1 except using a 10 ml test tube instead of the cornea-preserving container, and the corneal limbuses were transported. The transported corneal limbuses of Samples A and B were washed with PBS at 4° C. DMEM was dissolved at 4° C. to generate a 10% TGP solution, 10 ml of which was added to 25 cm² flasks each containing a part of tissue slices of Samples A and B, which was then pipetted to uniformly disperse the tissue slices. To this, 7 to 8 ml of 10% serum-containing DMEM was added, and this was cultured in 5% carbon dioxide incubator for 14 days. The proliferative ability of the tissue in culture was observed using a inverted microscope (10× magnification). The results are shown in FIGS. 6A and 6B.

In Sample B, the proliferation of cells from the graft was confirmed on Day 7. On the other hand, in Sample A, a good proliferation of cells was confirmed on Day 2. From this, it was found that the transportation using the TGP would cause less damage to the corneal limbus than transporting with MK medium. Accordingly, it was elucidated that the TGP is suitable for transporting corneal limbus.

### Example 5: Transportation of Intestinal Tissue

From a human large intestine resected by enterectomy for Hirschsprung's disease, 3 mm³ of intestine tissue was collected, cut into tissue slices. These were immersed in PBS containing antibiotics (gentamicin (50 μg/ml), amphotericin (0.25 μg/ml), penicillin (100 Units/ml)/streptomycin (100 μg/ml)) for 30 minutes. Each tissue slice was twice centrifuged and washed at 4° C. in PBS. Each tissue slice was equally cut into two pieces, each added into one of two 10 ml test tube (Test Tubes A and B).

Sample A: One gram of the TGP produced in Production Example was dissolved in 9 ml of DMEM/F12 at 4° C. to generate a 10% TGP solution, of which 10 ml was added to Test Tube A containing a tissue slice. After gelation at 30° C. for 1 hour, this was transported at a temperature varying between 5 and 42° C. (outdoor temperature) for 2 hours.

Sample B: Ten milliliter of PBS was added to Test Tube B containing a tissue slice, which was transported at 4° C. for 2 hours.

The tissue slices of Samples A and B were washed with 4° C. PBS, then each treated with Trypsin-EDTA solution (0.25%) at 37° C. for 30 minutes, then digested with Collagenase II solution (1 mg/ml) at 37° C. for 19 hours. After being filtrated through a 100 μm cell-strainer, it was centrifuged (150 rpm, 5 minutes). To this, a solution of TGP (dissolved in 9 ml of DMEM/F12 at 4° C.) (Mebiol Inc., 25 cm² flask) was added, and the cells were uniformly dispersed. This was added to a 25 cm² flask, gelled at 30° C. for 1 hour. To this, 7 to 8 ml of DMEM/F12 was added, which was then cultured in 5% carbon dioxide incubator for 2 weeks (Sample A-TGP, Sample B-TGP). The proliferative ability of the cells in culture was confirmed using an inverted microscope (10× magnification), then the number of the viable cells was measured using trypan blue. The observation results are shown in FIGS. 7A and 7B. In Sample A, enteric neuronal stem cells proliferated well as neurosphere-like bodies, whereas the proliferation was poor in Sample B. The number of cells after culturing was about 20 to 30 times higher in Sample A as compared with in Sample B. From this, it was elucidated that the TGP is suitable for transporting an intestinal tissue.

### Example 6: Transportation of Vascular Tissue

A saphenous vein tissue was collected from human body remains, which was cut into tissue slices. This was immersed in PBS containing antibiotics (gentamicin (50 μg/ml), amphotericin (0.25 μg/ml), penicillin (100 Units/ml)/streptomycin (100 μg/ml)) for 30 minutes. Each tissue slice was twice centrifuged and washed at 4° C. in PBS. Each tissue slice was equally cut into two pieces, each added into one of the two 10 ml test tube (Test Tubes A and B).

Sample A: One gram of the TGP produced in Production Example was dissolved in 9 ml of M199 at 4° C. to generate a 10% TGP solution, of which 10 ml was added to Test Tube A containing a tissue slice. After gelation at 30° C. for 1 hour, this was transported at a temperature varying between 5 and 42° C. (outdoor temperature) for 24 hours.

Sample B: Ten milliliter of HBSS was added to Test Tube B containing a tissue slice, which was transported at 4° C. for 2 hours.

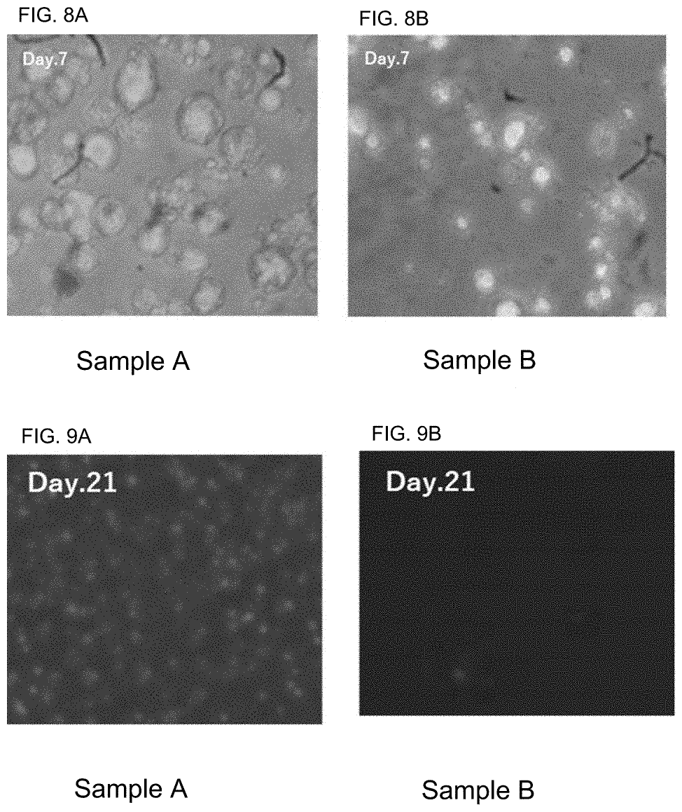

The tissue slices of Samples A and B were washed with 4° C. PBS, then each treated with Trypsin-EDTA solution (0.25%) at 37° C. for 30 minutes, then digested with Collagenase II solution (1 mg/ml) at 37° C. for 19 hours. After being filtrated through a 100 μm cell-strainer, it was centrifuged (150 rpm, 5 minutes), a solution of the TGP dissolved in 9 ml of M199 at 4° C. (Mebiol Inc., 25 cm² flask) was added, and the cells were uniformly dispersed. This was added to a 25 cm² flask, gelled at 30° C. for 1 hour. To this, 7 to 8 ml 10% serum-containing M199 was added, which was culture in 5% carbon dioxide incubator for 1 week (Sample A-TGP, Sample B-TGP). The proliferative ability of the cells in culture was confirmed using an inverted microscope (10× magnification), then the number of the viable cells was measured using trypan blue. The observation results are shown in FIGS. 8A and 8B.

In Sample A, a good proliferation was observed with relatively large and good cell shape. In Sample B, there was poor proliferation with small cell morphology. From this, it was elucidated that the TGP is suitable for transporting a vascular tissue.

### Example 7: Transportation of Dental Pulp Tissue

Dental pulp tissues were collected according to the same procedures as in Example 6, except that they were collected from 20 detached deciduous incisors, molars and cuspids obtained from 15 healthy human subjects instead of collecting a saphenous vein tissue from human body remains.

Sample A: One gram of the TGP produced in Production Example was dissolved in 9 ml of DMEM at 4° C. to generate a 10% TGP solution, of which 10 ml was added to Test Tube A containing a tissue slice. After gelation at 30° C. for 1 hour, this was transported at a temperature varying between 5 and 42° C. (outdoor temperature) for 24, 48 or 96 hours.

Sample B: Ten milliliter of PBS was added to Test Tube B containing a tissue slice, which was transported at 4° C. for 2 hours.

The tissue slices of Samples A and B were washed with 4° C. PBS, then centrifuged (150 rpm, 5 minutes). To this, a solution of the TGP dissolved in 9 ml of DMEM at 4° C. (Mebiol Inc., 25 cm² flask) was added, and the dental pulp tissues were uniformly dispersed. This was added to a 25 cm² flask, gelled at 20° C. for 1 hour. To this, 7 to 8 ml of 10% serum-containing DMEM was added, which was cultured in 5% carbon dioxide incubator for 2 weeks. The proliferative ability of the cells in culture was confirmed using an inverted microscope (10× magnification). The results are shown in FIGS. 9A and 9B.

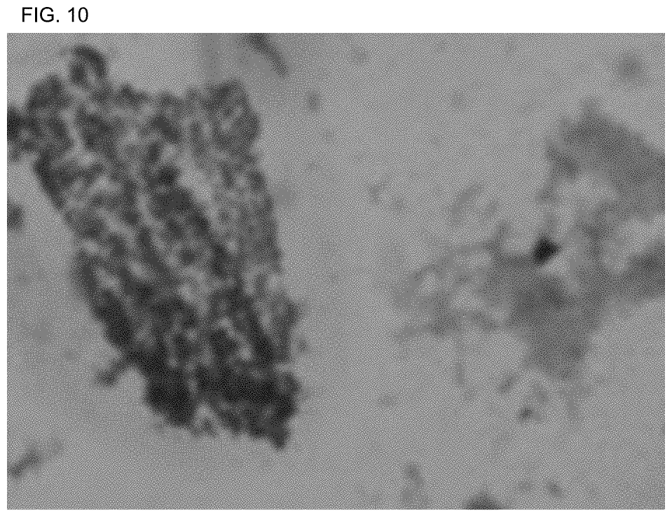

The dental pulp tissues in Samples A and B were also stained with H & E after being cultured for 21 days. The H & E staining of the dental pulp tissue of Sample A is shown in FIG. 10.

By observation using an inverted microscope, a better cell proliferation from the cultured tissue was confirmed in Sample A as compared with in Sample B. Moreover, healthy dental pulp tissue was confirmed by H & E in Sample A on Day 21, though no stained dental pulp tissue was confirmed in Sample B. From this, it was elucidated that the TGP is suitable for transporting a dental pulp tissue.

### Example 8: Preservation of Prepuce Tissue

From a circumcised human penis, 1 cm² of prepuce tissue was collected, which was cut into tissue slices. This was immersed in PBS containing antibiotics (gentamicin (50 μg/ml), amphotericin (0.25 μg/ml), penicillin (100 Units/ml)/streptomycin (100 μg/ml)) for 30 minutes. Each tissue slice was twice centrifuged and washed at 4° C. in PBS. Each tissue slice was equally cut into two pieces, each added into one of the two 10 ml test tube (Test Tubes A and B).

Sample A: One gram of the TGP produced in Production Example was dissolved in 9 ml of DMEM at 4° C. to generate a 10% TGP solution, of which 10 ml was added to Test Tube A containing a tissue slice. After gelation at 30° C. for 1 hour, this was left still at a temperature varying between 5 and 42° C. (outdoor temperature) for 24 hours for preservation.

Sample B: Ten milliliter of Hanks' balanced saline solution (HBSS) was added to Test Tube B containing a tissue slice, which was left still at 4° C. for 24 hours for preservation.

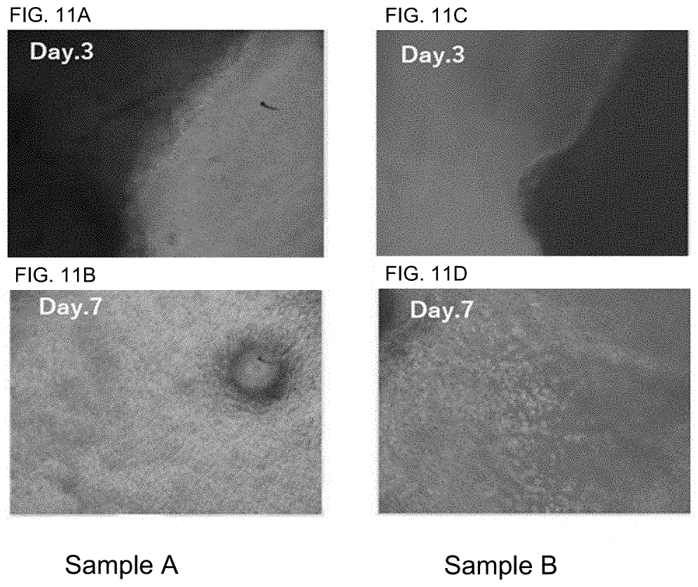

The tissue slices of Samples A and B were washed with 4° C. PBS, then each treated with Trypsin-EDTA solution (0.25%) at 37° C. for 30 minutes, then digested with Collagenase II solution (1 mg/ml) at 37° C. for 19 hours. After being filtrated through a 100 μm cell-strainer, it was centrifuged (150 rpm, 5 minutes). To this, a solution of the TGP dissolved in 9 ml of DMEM at 4° C. (Mebiol Inc., 25 cm² flask) was added, and the cells were uniformly dispersed. This was added to a 25 cm² flask, gelled at 30° C. for 1 hour. To this, 7 to 8 ml of 10% serum-containing DMEM was added, which was cultured in 5% carbon dioxide incubator for 2 weeks. The proliferative ability of the cells in culture was confirmed using an inverted microscope (10× magnification), then the number of the viable cells was measured using trypan blue. Microscopic images are shown in FIGS. 11A-11D.

In Sample A, as compared with Sample B, the cells emerged from the tissue to start growing, and there were 5 to 8 times more cells than Sample B on Day 7. From this, it was elucidated that the TGP is suitable for transporting a skin tissue.

### Example 9: Transportation and Culture of Greater Omental Tissue

Samples were prepared according to the same procedures as in Example 8, except that 2 to 3 cm² of greater omental tissues were collected from human body remains instead of prepuce tissue and that M199 was used instead of DMEM, and transported under following conditions:

Sample A: Transported at a temperature varying between 5 and 42° C. (outdoor temperature) for 12 hours; and,

Sample B: Transported at 4° C. for 12 hours.

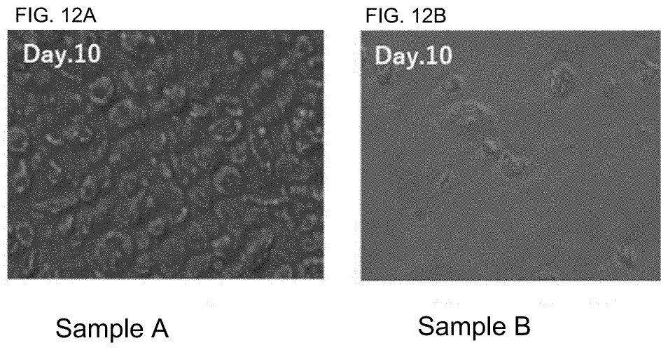

The tissue slices of Samples A and B were washed with 4° C. PBS, then each treated with Trypsin-EDTA solution (0.25%) at 37° C. for 30 minutes, then digested with Collagenase II solution (1 mg/ml) at 37° C. for 19 hours. After being filtrated through a 100 μm cell-strainer, it was centrifuged (150 rpm, 5 minutes). To a flask containing a TGP solution generated by adding 9 ml of M199 to a TGP solution (Mebiol Inc., 25 cm² flask), dispersed cells of Sample A were added and uniformly dispersed. This was added into a 25 cm² flask, gelled at 30° C. for 1 hour. To this, 7 to 8 ml of 10% serum-containing M199 was added, which was cultured in 5% carbon dioxide incubator for 10 days. To the dispersed cells of Sample B, 10 ml of M199 was added, which was cultured in 5% carbon dioxide incubator for 2 weeks. The proliferative ability of the cells in culture was confirmed on Day 10 using an inverted microscope (10× magnification). The results are shown in FIGS. 12A and 12B.

In Sample A, there was a better cell growth as compared with Sample B, and about 15 to 20 times more cells in cell number were observed. From this, it was elucidated that the TGP is suitable for transporting a greater omental tissue.

### Example 10: Transportation of Human Fetus Hepatic Cell

Three each of Samples A and B were prepared according to the same procedures as in Example 9, except using six hepatic tissue slices of 2 to 3 cm² from collected from human fetus instead of the greater omental tissue, and using DMEM/HAM F-12 instead of M199, and transported under following conditions:

Sample A: Transported at a temperature varying between 5 and 42° C. (outdoor temperature) for 4 to 8 hours; and,

Sample B: Transported at a temperature varying between 5 and 42° C. (outdoor temperature) for 4 to 8 hours.

The tissue slices of Samples A and B were washed with 4° C. PBS, then each treated with Trypsin-EDTA solution (0.25%) at 37° C. for 30 minutes, then digested with Collagenase II solution (1 mg/ml) at 37° C. for 19 hours. After being filtrated through a 100 μm cell-strainer, it was centrifuged (150 rpm, 5 minutes) and suspended in 2 ml PBS. From each test tube, 100 μl was separately collected, to which 400 μL of 0.4% trypan blue solution was added, cells were counted using a cytometer. The results are shown in Table 4.

TABLE 4

| | Cell Count after | |
|---|---|---|
| | Transportation | |
| Sample | (×10⁶) | |
| No. | Sample A | Sample B |
| 1 | 0.16 | 0.06 |
| 2 | 0.23 | 0.09 |
| 3 | 0.56 | 0.13 |

As compared with PBS, when being transported using the TGP, high viable cell counts were confirmed in all cases.

From this, the TGP is capable of transporting a hepatic tissue without damaging it. Accordingly, it was elucidated that the TGP is suitable for transporting a hepatic tissue.

From the results of Examples 1 to 10, it was found that the TGP is suitable for preservation and transportation of various biomaterials.

1. A biomaterial preserving composition comprising a thermoreversible gelation polymer.

2. The preserving composition according to claim 1, for preservation at a constant or varying temperature.

3. The preserving composition according to claim 2, wherein the constant or varying temperature is a temperature at which a cell does not substantially proliferate.

4. The preserving composition according to claim 3, wherein the temperature at which a cell does not substantially proliferate is between 4 and 30° C.

5. The preserving composition of claim 1, wherein the biomaterial is selected from the group consisting of a cartilage tissue, oral mucosa tissue, corneal tissue, corneal limbus tissue, dental pulp tissue, vascular tissue, gastrointestinal mucosa tissue, greater omentum tissue, skin tissue and hepatic tissue.

6. The preserving composition of claim 1, wherein the biomaterial is a somatic cell, precursor cell or stem cell contained in a tissue selected from the group consisting of a cartilage tissue, oral mucosa tissue, corneal tissue, corneal limbus tissue, dental pulp tissue, vascular tissue, gastrointestinal mucosa tissue, greater omentum tissue, skin tissue and hepatic tissue.

7. The preserving composition of claim 1, wherein the thermoreversible gelation polymer comprising a plurality of blocks having cloud point selected from the group consisting of polypropylene oxide, a copolymer of propylene oxide and (an)other alkylene oxide(s), a poly N-substituted acrylamide derivative, a poly N-substituted methacrylamide derivative, a copolymer of an N-substituted acrylamide derivative and an N-substituted methacrylamide derivative, polyvinylmethylether and partially acetylated polyvinyl alcohol, and a hydrophilic block bound thereto.

8. The preserving composition of claim 1, wherein the preserving composition can also be used for transportation.


SEQUENCE LISTING (4 sequences)

SEQ ID NO: 1 - DNA, 25 bp, Artificial Sequence
  Foward primer for SOX9
ggagatgaaatctgttctgggaatg

SEQ ID NO: 2 - DNA, 25 bp, Artificial Sequence
  Reverse primer for SOX9
ttgaaggttaactgctggtgttctg

SEQ ID NO: 3 - DNA, 22 bp, Artificial Sequence
  Forward primer for COL2A1
ccagttgggagtaatgcaagga

SEQ ID NO: 4 - DNA, 21 bp, Artificial Sequence
  Reverse primer for COL2A1
acaccaggttcaccaggttca